(19) United States
(12) Patent Application Publication
(10) Pub. No.: US 2010/0197702 A1
(43) Pub. Date: Aug. 5, 2010

(54) OPHTHALMIC COMPOSITION WITH NITRIC OXIDE DONOR COMPOUND AND METHOD OF FORMING AND USING SAME

(76) Inventors: Mark R. Hellberg, Arlington, TX (US); Peter G. Klimko, Fort Worth, TX (US); Jesse A. May, Fort Worth, TX (US); Suchismita Mohapatra, Arlington, TX (US)

Correspondence Address: ALCON, IP LEGAL, TB4-8, 6201 SOUTH FREEWAY, FORT WORTH, TX, 76134, US

(21) Appl. No.: 12/694,417

(22) Filed: Jan. 27, 2010

Related U.S. Application Data
(60) Provisional application No. 61/149,723, filed on Feb. 4, 2009.

(51) Int. Cl. A61K 31/4468; A61K 31/4015; A61P 27/02; A61K 31/454; A61K 31/496

(52) U.S. Cl. 514/252.18; 514/315; 514/425; 514/326

(57) ABSTRACT

The present invention is directed to the provision of ophthalmic compositions such as multi-dose, topical, ophthalmic compositions. The compositions include a nitric oxide (NO) donor compound.

### CROSS REFERENCE TO RELATED APPLICATION

This application claims priority under 35 U.S.C. §119 to U.S. Provisional Patent Application No. 61/149,723, filed Feb. 4, 2009, the entire contents of which are incorporated herein by reference.

### TECHNICAL FIELD OF THE INVENTION

The present invention is related to an ophthalmic composition that includes at least one nitric oxide donor compound. More particularly, the present invention is related to an ophthalmic composition that includes a nitric oxide donor pyrrolidone or piperidine N-oxyl free radical for the treatment of age-related macular degeneration (AMD), diabetic retinopathy (DR), high intraocular pressure (TOP), and uveitis.

### BACKGROUND OF THE INVENTION

Nitric oxide (NO) is a gaseous molecule that is biosynthesized via an enzyme-catalyzed reaction between molecular oxygen and the amino acid arginine. The enzyme, called nitric oxide synthase (NOS), has three isoforms that have been characterized to date: eNOS, which is primarily expressed in the endothelium; nNOS, which is primarily expressed in neurons; and iNOS, which is primarily expressed in white blood cells. NO plays an important role as an intra- and intercellular messenger in the cardiovascular, nervous, and immune systems.

NO derived from endothelium and efferent nitrergic neurons has been reported to regulate ocular blood flow, with endothelial dysfunction due to increased production of reactive oxygen species (ROS) impairing ocular hemodynamics. In particular, enhanced superoxide production may reduce NO bioavailability by converting it to the toxic ROS peroxynitrite [Toda et al. Nitric oxide: Ocular blood flow, glaucoma, and diabetic retinopathy. Progress in Retinal and Eye Research, 2007, 26, 205-238].

Chiou has suggested that DR, AMD, and glaucomatous optic neuropathy are all associated with enhanced oxidative stress. Inhibition of oxidative stress-induced nitric oxide destruction was hypothesized to allow preservation of nitric oxide's neuroprotective role [Chiou, G. C. Neuroprotective properties of Nitric oxide. Annals of the New York Academy of Science 1999, 215, 113-116].

It has also recently been suggested that uncoupled NOS, due to limited availability of substrate arginine or co-factor tetrahydrobiopterin, is likely a major source of superoxide in diabetic retinal endothelial cells and results in an increased concentration of peroxynitrite. Pathological effects attributed to peroxynitrite in endothelial cells include induction of VEGF protein production [Platt et al. Peroxynitrite increases VEGF expression in vascular endothelial cells via STAT3 Free Radicals in Biology and Medicine. 2005, 39(10), 1353-1361] and inactivation of VEGF survival signaling [el-Remessy et al. Oxidative stress inactives VEGF survival signaling in retinal endothelial cells via PI 3-kinase tyrosine nitration. Journal of Cell Science 2005, 118(Pt 1), 243-252].

High glucose concentration has been reported to induce NOS uncoupling and increase protein nitration in retinal endothelial cells. These effects were reversed by inhibiting aldose reductase or eNOS, adding supplemental arginine or tetrahydrobiopterin, or scavenging superoxide or peroxynitrite [el-Remessy et al. High glucose-induced tyrosine nitration in endothelial cells: role of eNOS uncoupling and aldose reductase activation. Investigative Opthalmology and Visual Science 2003, 44(7), 3135-3143].

In human microvascular endothelial cells, Selemidis et al. reported that NO was reported to suppress NADPH oxidase-induced superoxide production by S-nitrosylating an unidentified cysteine thiol in the p47phox subunit of NADPH oxidase [Selemidis, S.; Dusting, G. J.; Peshavariya, H.; Kemp-Harper, B. K.; Drummond, G. R. Nitric oxide suppresses NADPH oxidase-dependent superoxide production by S-nitrosylation in human endothelial cells. Circulation 2007, 75(2), 349-358]. How this inhibits the enzyme's activity was not disclosed. Similarly, Park has disclosed that S-nitrosothiols such as S-nitrosoglutathione inhibit NADPH oxidase subunits p47^{phox} and p67^{phox} membrane translocation in neutrophils, in a mercaptoethanol-reversible manner [Park, J. W. Biochemical Biophyical. Research Communications 1996, 220, 31-35].

Generally, it is believed that early development of nitric oxide tolerance is a major drawback in NO-donor based therapies. Moreover, several recent reports indicate superoxide as having a significant role in mediation of such tolerance [Circulation Research, 1994, 74, 1141-1148].

Other recent articles have disclosed nitrates of TEMPOL (compound 1 below) and of hydroxymethyl-Proxyl (NMP, compound 2 below) as vasodilators which do not produce NO-tolerance in vivo, possibly because of their ability to detoxify superoxide [Gasco, A, Fruttero, R, Rolando, B; Focus on recent approaches for the development of new NO-donors. Mini-Reviews in Medicinal Chemistry, 2005, 5, 217-229. Haj-Yehia, A, Nassar, T, Lotan, C, Munzel, T, Benet, L, and Anggard, E. E. Development of 3-Nitratomethyl-Proxyl (NMP): A novel, bifuntional Superoxide Dismutase-mimic-Nitric oxide-donor. Drug Development Research, 2000, 50, 528-536].

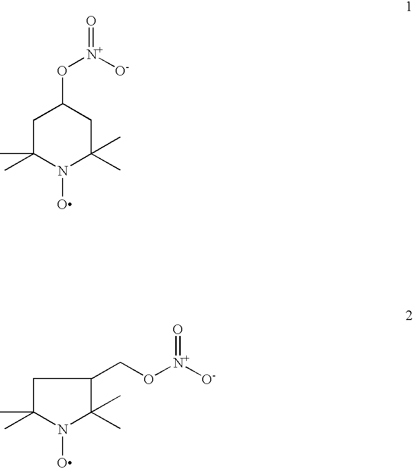

WO 99/37616 (Anngard et al.) disclosed a method of preparation of NO-donating piperidine and pyrroline N-oxyl free radicals, such as 1 and 2 above. Compound 1 was assessed for its superoxide scavenging activity, vasorelaxation efficacy, effect on c-GMP production, and effect on tolerance to organic nitrates. The in vitro vasorelaxation assay demonstrated the superior activity of compound 1 over the benchmark organic nitrate NO donors glyceryl tri-nitrate (GTN) and S-nitroso-N-acetylpenicillamine (SNAP). Also, GTN induced tolerance both to itself and to 1, while 1 did not induce tolerance to itself or to GTN. Moreover, compound 1 afforded a significant increase in c-GMP concentration. Although it bears only one nitrate group, the effect of 1 was comparable to that of GTN, suggesting that the ROS scavenging ability of 1 may play a role in enhancing NO bioavailability.

Patil and Mousa (WO 2008/101195 A2) discloses the use of certain N-hydroxy-piperidines and -pyrrolidines for the treatment of AMD. Compound 3 below is the only NO-donating N-hydroxypiperidine or pyrrolidine disclosed in the application.

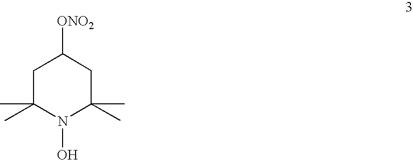

No piperidin-N-oxyl or pyrrolidin-N-oxyl free radicals are suggested. Furthermore, in paragraph [0032] it is stated that “Due to their comparative lack of toxicity, hydroxylamines are preferable to nitroxides as therapeutic agents.” Thus Patil and Mousa suggest that a piperidin-N-oxyl or pyrrolidin-N-oxyl free radical is not suitable as a therapeutic agent for the treatment of ocular diseases, even if the corresponding N-hydroxy compound is.

Although the art has provided information about the potential therapeutic effects of NO and/or some NO donor compounds, it also suggests that excess NO production can have untoward effects on ocular tissue. For example, the iNOS isoform is up-regulated by pro-inflammatory cytokines like TNF-α and IL-1β in a variety of cell types, and can produce super-physiological concentrations of NO. Subsequent conversion to species like NO₂ and nitrite may contribute to AMD and DR pathological progression [Chiou, G. C. Review: effects of nitric oxide on eye diseases and their treatment. Journal of Ocular Pharmacology and Therapeutics 2001, 17(2), 189-198]. Other disclosures suggest that NO production is increased in diabetic vs. normal animals [Yunpeng, D.; Sarthy, V. P.; Kern. T. S. Interaction between NO and COX pathways in retinal cells exposed to elevated glucose and retina of diabetic rats. American Journal of Physiology 2004, 287(4, Part 2), R735-R741], is pro-angiogenic in the retina and choroid [Ando, A.; et al. Nitric oxide is pro-angiogenic in the retina and choroid. Journal of Cellular Physiology 2002, 191(1), 116-124], is increased by VEGF in bovine choroidal epithelial cells with enhancement of endothelial cell migration and proliferation [Uhlmann, S.; et al. Direct measurement of VEGF-induced nitric oxide production by choroidal endothelial cells. Microvascular Research 2001, 62, 179-189], is increased in the plasma of humans with proliferative diabetic retinopathy [Tsai, D.-C.; et al. Different plasma levels of vascular endothelial growth factor and nitric oxide between patients with choroidal and retinal neovascularization. Opthalmologica 2006, 220(4), 246-251], and plays a role in anterior chamber uveitis pathology [Allen, J. B.; Keng, T.; Privalle, C. Nitric oxide and peroxynitrite production in ocular inflammation. Environmental Health Perspectives. 1998, 106, Supplement 5, 1145-1149]. Thus, since the art suggests that NO may be a pathological factor in AMD, DR, glaucoma, and uveitis, it is not clear that NO-donating compounds as a class, and NO-donating pyrrolidin- or piperidin-N-oxyl free radicals in particular, represent a general solution to the treatment of these ocular diseases.

Based on the art, it is difficult to identify or otherwise provide nitric oxide donor compounds useful for ophthalmic compositions. In particular, it is difficult to identify particular nitric oxide donor compounds suitable for use and delivery as part of ophthalmic compositions. Moreover, it is difficult to identify particular nitric oxide donor compounds that are likely to be efficacious in ameliorating the symptoms, effects or causes of various ophthalmic diseases such as high IOP, DR uveitis, and wet and dry AMD. Therefore, the present disclosure is directed to nitric oxide donor compounds useful in treating ophthalmic diseases and ophthalmic compositions including those compounds as well as methods of making and/or using the compositions.

### SUMMARY OF THE INVENTION

Accordingly, there is disclosed ophthalmic compositions containing particular nitrato piperidine and pyrroline nitroxyl and N-hydroxylamine compounds that are useful for the topical treatment of several ocular diseases, such as AMD, diabetic retinopathy, uveitis, and high intraocular pressure (IOP).

According to one aspect of the present invention there is provided an ophthalmic composition comprising a compound of formula A:

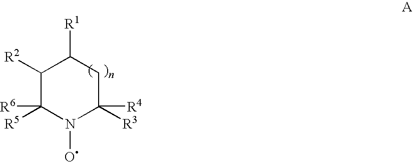

wherein:
R¹ is an NO donor group, such as ONO₂, CH₂ONO₂, D¹, D², or D³;

### D¹ is OC(═O)X¹;

### X¹ is L¹ or L²;

### L¹ is

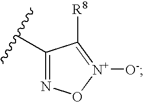

### R⁸ is CN or C(O)NH₂;

### L² is

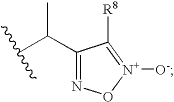

### D² is C(O)X²;

### X² is OL¹, OL², OL⁴, L⁵, or L⁶;

### L⁴ is

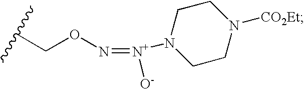

### L⁵ is

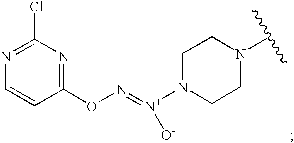

### L⁶ is

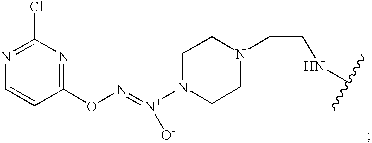

### D³ is NH—X³;

### X³ is CH₂L¹, CH₂L², C(O)L¹, C(O)L², or L⁷;

### L⁷ is

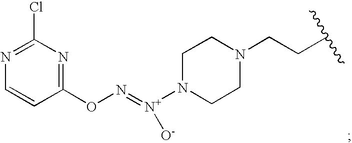

### R² is H, OH, or OC(O)R⁷;

R⁷ is C₁₋₈ alkyl, C₃₋₈ cycloalkyl, benzyl, or phenyl;
n is 0 or 1;
R³, R⁴, R⁵, and R⁶ are the same or different and are C₁-C₈ alkyl or C₃-C₈ cycloalkyl; and
a suitable ophthalmic vehicle.

In one particularly preferred embodiment, R² is H; and R³, R⁴, R⁵, and R⁶ are all CH₃. Such compound can be selected from the following compounds:

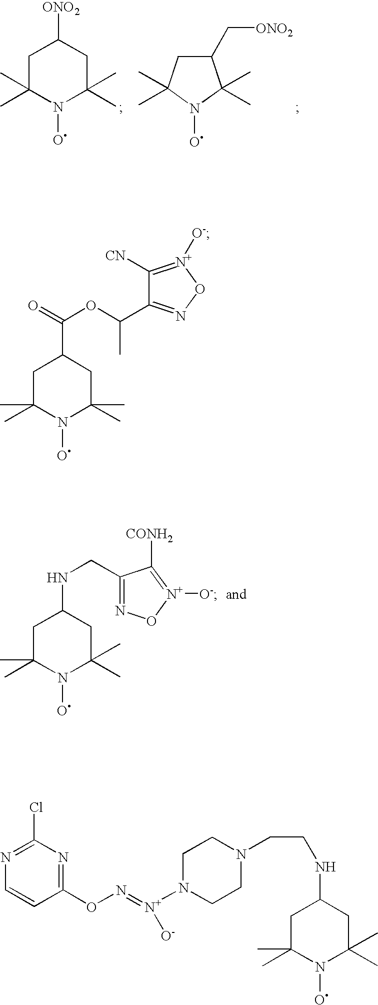

The composition can be formulated for topical application and can be disposed with an eye dropper. The composition can include an antimicrobial agent, a surfactant, a tonicity agent or a combination thereof. Moreover, the composition can have a pH in the range of 4 to 9, preferably 5.5 to 8.5, and most preferably 5.5 to 8.0. Particularly desired pH ranges are 6.0 to 7.8 and more specifically 6.4 to 7.6. Furthermore, the composition can have an osmolality of 200 to 400 or 450 milliosmoles per kilogram (mOsm/kg), more preferably 240 to 360 mOsm/kg.

### DETAILED DESCRIPTION OF THE INVENTION

Unless otherwise stated, percentages for ingredients of the ophthalmic composition of the present invention are weight/volume percentages (w/v %).

The present invention encompasses an ophthalmic composition that includes a nitric oxide (NO) donating compound of formula A:

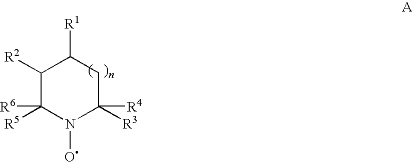

wherein:
R¹ is an NO-donating group, such as NO₂, CH₂ONO₂, D¹, D², or D³;

### D¹ is OC(═O)X¹;

### X¹ is L¹ or L²;

### L¹ is

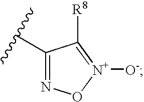

### R⁸ is CN or C(O)NH₂;

### L² is

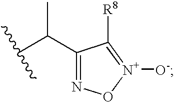

### D² is C(O)X²;

### X² is OL¹, OL², OL⁴, L⁵, or L⁶;

### L⁴ is

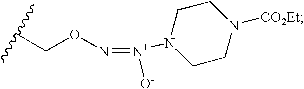

### L⁵ is

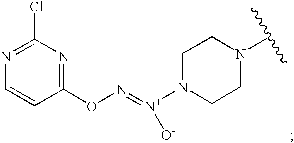

### L⁶ is

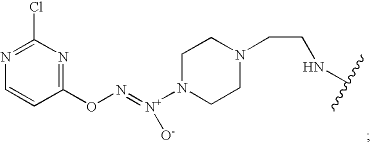

### D³ is NH—X³;

### X³ is CH₂L¹, CH₂L², C(O)L¹, C(O)L², or L⁷;

### L⁷ is

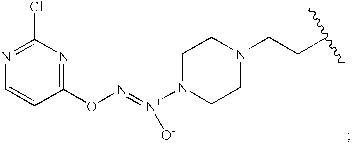

### R² is H, OH, or OC(O)R⁷;

R⁷ is C₁₋₈ alkyl, C₃₋₈ cycloalkyl, benzyl, or phenyl;
n is 0 or 1; and
R³, R⁴, R⁵, and R⁶ are the same or different and are C₁-C₈ alkyl or C₃-C₈ cycloalkyl.

Generally, an NO donor group is defined herein as being a chemical moiety that releases an NO group upon exposure to an eye of a mammal, particularly a human. The NO donor group can be selected from a variety of chemical moieties. Potentially suitable moieties include, without limitation, organic nitrates, sydnomines, furoxans and diazenium diolates.

Preferred compound of formula A for ophthalmic compositions and methods of use are those wherein:

### R² is H; and

### R³, R⁴, R⁵, and R⁶ are all CH₃.

Among the most preferred are the following compounds 1-5 below.

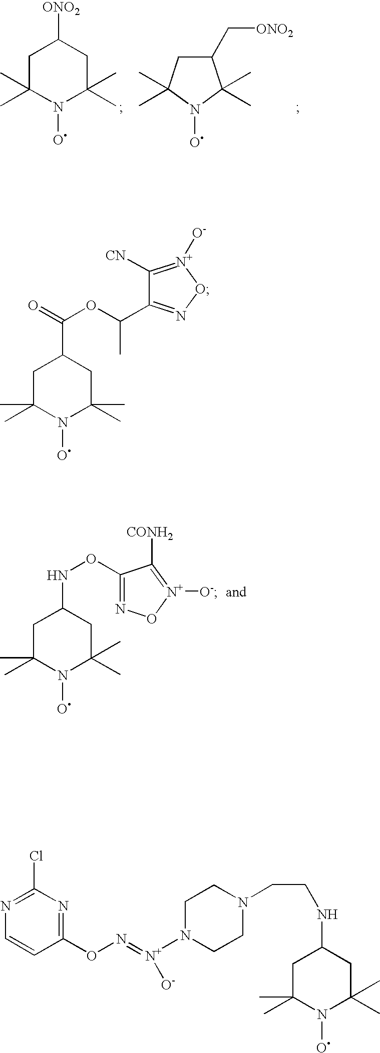

The NO donor compound will typically be a small percentage of the total ophthalmic composition. The NO donor compound will typically be at least 0.01 w/v %, more typically at least 0.1 w/v % and even more typically at least 0.5 w/v % of the ophthalmic composition. The NO donor compound will also typically be no greater than 5.0 w/v %, even more typically no greater that 3.0 w/v % and even more typically no greater than 1.5 w/v % of the ophthalmic composition.

The ophthalmic composition will also typically include a suitable ophthalmic vehicle for delivery of the compound to the eye. It is contemplated that the ophthalmic composition may be configured for topical or intravitreal application to the eye and the ophthalmic vehicle will likely be different depending upon the manner of application. Generally, for either topical or intravitreal applications, it is preferable that the ophthalmic composition be aqueous and include a substantial amount of water. Typically the composition will include at least 30 w/v %, more typically at least 80 w/v % and even more typically at least 90 w/v % water (e.g., purified water).

For intravitreal applications, particularly when the ophthalmic composition is applied to the eye with a syringe, the ophthalmic compositions may include only or consist essentially of water and the NO donor compound. Of course the ophthalmic composition could include other ingredients as well such as Na₂HPO₄, hydroxypropyl methylcellulose, polysorbate 80, sodium chloride, and edentate disodium.

It could also be the case that the vehicle be only or consist essentially of water for a topical application, particularly if that topical application is performed shortly after water is combined with the NO donor compound or the composition is packaged in a manner to prevent contamination. However, if the ophthalmic composition is to be applied as a multi-dose ophthalmic composition over an extended period of time (e.g., as drops from an eye-dropper once, twice, thrice or more per day for multiple days), the ophthalmic composition will likely include additional ingredients such as antimicrobial or preservative agents or systems, surfactants, buffering agents, tonicity agents, anti-oxidants, viscosity-modifying agents any combinations thereof or the like.

For topical application, the compositions of the present invention typically include antimicrobial agent. Potential antimicrobial agents include, without limitation, hydrogen peroxide, chlorine containing preservatives such as benzalkonium chloride or others. According to a preferred aspect, however, the composition of the present invention is entirely or substantially free of any non-polymeric quaternary anti-microbial agents such as benzalkonium chloride (BAK). Most preferred antimicrobial agent in the pharmaceutical composition includes polymeric quaternary ammonium compound.

As used herein, the phrase “substantially free of” as it refers to an ingredient of the ophthalmic composition means that it is contemplated that the ophthalmic composition can be either entirely devoid of that particular ingredient or includes only a nominal amount of that particular ingredient.

The polymeric quaternary ammonium compounds useful in the compositions of the present invention are those which have an antimicrobial effect and which are ophthalmically acceptable. Preferred compounds of this type are described in U.S. Pat. Nos. 3,931,319; 4,027,020; 4,407,791; 4,525,346; 4,836,986; 5,037,647 and 5,300,287; and PCT application WO 91/09523 (Dziabo et al.). The most preferred polymeric ammonium compound is polyquaternium 1, otherwise known as POLYQUAD™ or ONAMERM™ with a number average molecular weight between 2,000 to 30,000. Preferably, the number average molecular weight is between 3,000 to 14,000.

The polymeric quaternary ammonium compounds are generally used in the suspensions of the present invention in an amount that is greater than about 0.00001 w/v %, more typically greater than about 0.0003 w/v % and even more typically greater than about 0.0007 w/v % of the suspension. Moreover, the polymeric quaternary ammonium compounds are generally used in the compositions of the present invention in an amount that is less than about 3 w/v %, more typically less than about 0.003 w/v % and even more typically less than about 0.0015 w/v % of the composition.

The antimicrobial agent of the composition of the present invention can additionally or alternatively include an antimicrobial system such as a borate/polyol complex system. As used herein, the term “borate” shall refer to boric acid, salts of boric acid, borate derivatives and other pharmaceutically acceptable borates, or combinations thereof. Most suitable are: boric acid, sodium borate, potassium borate, calcium borate, magnesium borate, manganese borate, and other such borate salts. Borate interacts with polyols, such as glycerol, propylene glycol, sorbitol and mannitol, to form borate polyol complexes. The type and ratio of such complexes depends on the number of OH groups of a polyol on adjacent carbon atoms that are not in trans configuration relative to each other. It shall be understood that weight/volume percentages of the ingredients polyol and borate include those amounts whether as part of a complex or not.

As used herein, the term “polyol” includes any compound having at least one hydroxyl group on each of two adjacent carbon atoms that are not in trans configuration relative to each other. The polyols can be linear or cyclic, substituted or unsubstituted, or mixtures thereof, so long as the resultant complex is water soluble and pharmaceutically acceptable. Examples of such compounds include: sugars, sugar alcohols, sugar acids and uronic acids. Preferred polyols are sugars, sugar alcohols and sugar acids, including, but not limited to: mannitol, glycerin, xylitol, sorbitol and propylene glycol.

When used, the borate/polyol complex antimicrobial system (i.e., the borate and polyol together) typically comprise at least 0.05 w/v %, more typically at least 0.5 w/v % and even possibly at least 1 or even at least 1.2 w/v % of the composition and also typically comprise less than 5 w/v %, more typically less than 2.2 w/v % and even possibly less than 1.6 w/v % of the composition. The borate to polyol ratio (weight to weight ratio) in the composition is typically between 1 to 1 and 1 to 10 and more typically is between 1 to 2 and 1 to 4 (e.g., about 1 to 3).

Tyloxapol, polysorbate-80 and polyoxyl hydrogenated castor oil are preferred surfactants. Tyloxapol is a highly preferred surfactant. When used, the surfactant is typically present in a concentration that is at least 0.01 w/v %, more typically at least 0.025 w/v % and even possibly at least 0.1 w/v % of the composition and also typically is less than 5 w/v %, more typically less than 2.0 w/v % and even possibly less than 1.0 w/v % of the composition.

The compositions of the present invention that are to be used for topical applications are typically formulated so as to be compatible with the eye. The ophthalmic compositions intended for direct application to the eye will be formulated so as to have a pH and tonicity that are compatible with the eye. The compositions will typically have a pH in the range of 4 to 9, preferably 5.5 to 8.5, and most preferably 5.5 to 8.0. Particularly desired pH ranges are 6.0 to 7.8 and more specifically 6.4 to 7.6. The compositions will have an osmolality of 200 to 400 or 450 milliosmoles per kilogram (mOsm/kg), more preferably 240 to 360 mOsm/kg.

Preferred compositions of the present invention are multi-dose ophthalmic compositions, for example, where the composition is in an eye dropper and can be administered as one or more drops once, twice, thrice or more topically to the eye. In that case, the compositions preferably have sufficient antimicrobial activity to allow the compositions to satisfy the USP preservative efficacy requirements, as well as other preservative efficacy standards for aqueous pharmaceutical compositions.

The preservative efficacy standards for multi-dose ophthalmic solutions in the U.S, and other countries/regions are set forth in the following table:

Preservative Efficacy Test (“PET”) Criteria
(Log Order Reduction of Microbial Inoculum Over Time)

| | Bacteria | Fungi |
|---|---|---|
| USP 27 | A reduction of 1 log (90%), by day 7; | The compositions must demonstrate |
| | 3 logs (99.9%) by | over the entire test period, which means |
| | day 14; and no increase | no increases of 0.5 logs or greater, |
| | after day 14 | relative to the initial inoculum. |
| Japan | 3 logs by 14 days; and no | No increase from initial count at 14 and |
| | increase from day 14 | 28 days |
| | through day 28. | |
| Ph. Eur. A¹ | A reduction of 2 logs | A reduction of 2 logs (99%) by 7 days, |
| | (99%) by 6 hours; 3 logs by | and no increase thereafter |
| | 24 hours; and no recovery | |
| | after 28 days | |
| Ph. Eur. B | A reduction of 1 log at 24 | A reduction of 1 log (90%) by day 14, |
| | hours; 3 logs by day 7; and | and no increase thereafter |
| | no increase thereafter | |
| FDA/ISO | A reduction of 3 logs from | No increase higher than the initial value |
| 14730 | initial challenge at day 14; | at day 14, and no increase higher than |
| | and a reduction of 3 logs | the day 14 rechallenge count through |
| | from rechallenge | day 28. |

¹There are two preservative efficacy standards in the European Pharmacopoeia “A” and “B”.

The standards identified above for the USP 27 are substantially identical to the requirements set forth in prior editions of the USP, particularly USP 24, USP 25 and USP 26.

As an added advantage, these ophthalmic compositions containing NO donor compounds of the present invention are suitable for topical applications to the eye.

In addition to the above, IOP-lowering ophthalmic compositions comprising these NO donor compounds may also contain other therapeutic agents. Examples of such other therapeutic agents include, without limitation: prostaglandin analogs like latanoprost, bimatoprost, and travoprost; carbonic anhydrase inhibitors like dorzolamide and brinzolamide; β-adrenergic receptor antagonists like timolol and betaxolol; and α-adrenergic receptor agonists like brimonidine.

Advantageously, the compositions of the present invention can be particularly desirable for lowering intraocular pressure (IOP) of mammals. Thus, in one embodiment of the present invention, the compositions of the present invention are used therapeutically to lower IOP of a mammal such as a human being. In such therapeutic use or method, a test is typically performed for determining whether the mammal has elevated IOP. Of course, the skilled artisan will recognized that multiple tests exist for making such determination. Upon determination of elevated IOP, the composition of the present invention is then administered to the mammal either topically or intravitreally. For topical administration, an eye dropper of the composition is typically supplied to allow for self-administration.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

### Example 1

The table below represents exemplary ranges for a topical ophthalmic composition according to the present invention:

| Ingredients | w/v % |
|---|---|
| NO Donor Compound | 0.1 to 1.5 |
| Antimicrobial Agent | 0.1 to 5.0 |
| Surfactant | 0.001 to 1.0 |
| Tonicity Agent | 0.001 to 1.0 |
| Water | Q.S. to 100 |

### Example 2

Compound 1 was evaluated for its ability to prevent 7-ketocholesterol-induced retinal pigmented epithelial (RPE) cell death in vitro. ARPE-19 cells were grown in DMEM:F12 (Invitrogen) (1:1) with 10% FBS (HyClone) and 56 mM Na Bicarbonate (Gibco) in a 10% CO₂ humidified 37° C. incubator. Cells were split at a ratio of 1:3 once per week and fed every 2-3 days. ARPE-19 cells were plated at a density of 12,500 cells per well in 100 μL, which is approximately 0.5×10⁴ cells/cm².

7-ketocholesterol (5-CHOLESTEN-3β-OL-7-ONE) and Cholesterol (5-CHOLESTEN-3β-OL) (Steraloids, Inc., Newport R.I.) were resuspended in 37° C. 45% 2-hydroxylpropyl-β-cyclodextrin at a concentration of 10 mM. Working dilutions of 1 mM are subsequently made in 37° C. DMEM:/F12 containing 0.1% FBS and NaBicarb. Further dilutions (5 to 30 μM) for treatment were also made in 37° C. DMEM:F12 with 0.1% FBS and NaBicarb to maintain activity of the 7-ketocholesterol. As negative controls, vehicle alone and cholesterol suspended in 2-HP-β-cyclodextrin were included in each experiment.

Cell proliferation reagent WST-1 (Roche), a tetrazolium salt that is cleaved to formazan through the mitochondrial succinate-tetrazolium-reductase system in live cells, was used as a measure of cell viability. Cells were treated with 7-ketocholesterol for 1 day, at which time 10 μl, of WST-1 reagent was added to each well containing 100 μL of media. The plates were then incubated for 1-4 hours to allow for adequate color development and then absorbance was read in a microplate ELISA reader at 440 nM. WST reagent was also added to one set of control wells containing media only. These readings were subtracted from each of the WST readings as background. Cell viability for all treated wells was normalized to the well with untreated cells and data is presented as percent survival as compared to untreated. The table below summarizes the results.

| Agent | Concentration | | % Survival^{a} | p value^{b} |
|---|---|---|---|---|
| Media | — | | defined as 100% | — |
| 7-ketocholesterol | 20 | μM | 50% | — |
| 1 | 100 | nM | 60% | >0.05 |
| 1 | 1 | μM | 60% | >0.05 |
| 1 | 10 | μM | 75% | <0.05 |
| 1 | 100 | μM | 100% | <0.01 |

^{a}compared to media-treated cells.
^{b}by comparison to 7-ketocholesterol-treated cells, as calculated using ANOVA.

In summary, 10 μM of compound 1 provided significant, while 100 μM afforded complete, RPE cytoprotection.

### Example 3

The ability of compound 1 of the present invention to reduce IOP was evaluated in cynomolgus monkeys with ocular hypertension produced by previous laser trabeculoplasty in the right eye. Animals had been trained to sit in restraint chairs and conditioned to accept experimental procedures without chemical restraint. Animals were administered a 30 μl, drop containing 150 μg of compound 1 dissolved in vehicle to the lasered eye. IOP was determined with a pneumatonometer after light corneal anesthesia with dilute proparacaine. The drug was dosed 35 minutes after the baseline IOP was measured. The timecourse for the IOP effect of drug administration is summarized in the table below.

| % IOP change | Time after | |
|---|---|---|
| from baseline | dosing | |
| −15.3 | 1 | hour |
| −16.7 | 3 | hours |
| −20.7 | 6 | hours |

We claim:

1. An ophthalmic composition comprising a compound of formula A: 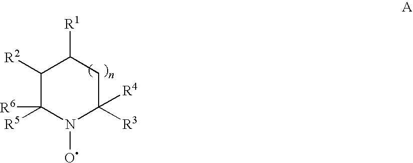
wherein:
R¹ is an NO donor group, such as ONO₂, CH₂ONO₂, D¹, D², or D³;
D¹ is OC(═O)X¹;
X¹ is L¹ or L²;
L¹ is 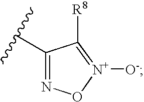
R⁸ is CN or C(O)NH₂;
L² is 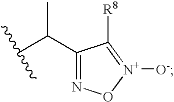
D² is C(O)X²;
X² is OL¹, OL², OL⁴, L⁵, or L⁶;
L⁴ is 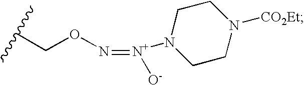
L⁵ is 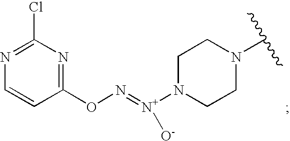
L⁶ is 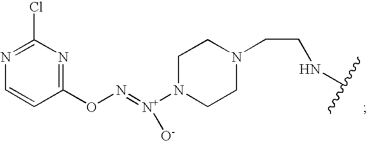
D³ is NH—X³;
X³ is CH₂L¹, CH₂L², C(O)L¹, C(O)L², or L⁷;
L⁷ is 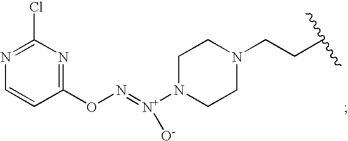
R² is H, OH, or OC(O)R⁷;
R⁷ is C₁₋₈ alkyl, C₃₋₈ cycloalkyl, benzyl, or phenyl;
n is 0 or 1;
R³, R⁴, R⁵, and R⁶ are the same or different and are C₁-C₈ alkyl or C₃-C₈ cycloalkyl; and
a suitable ophthalmic vehicle.

2. A composition as in claim 1, wherein:
R² is H; and
R³, R⁴, R⁵, and R⁶ are all CH₃.

3. A composition of claim 2, wherein the composition includes an antimicrobial agent and wherein the compound of formula A is selected from the group consisting of:

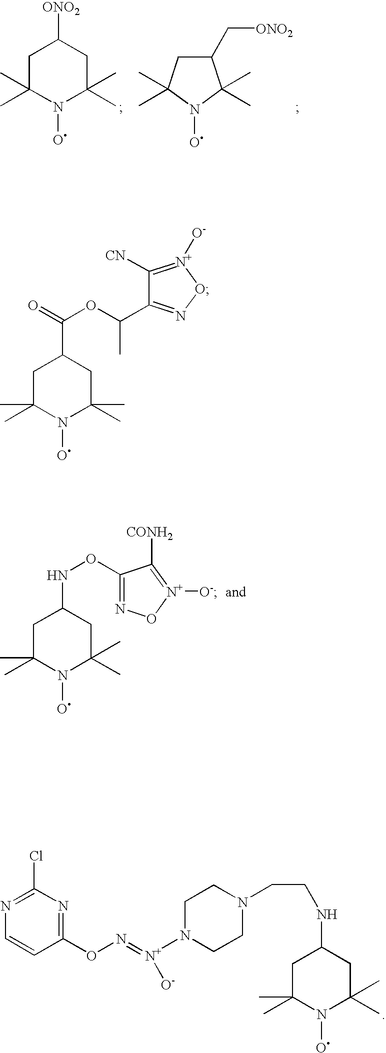

4. A composition as in claim 1 wherein the composition is formulated for topical application and is disposed with an eye dropper.

5. A composition as in claim 1 wherein the composition further includes an antimicrobial agent, a surfactant, a tonicity agent or a combination thereof.

6. A composition as in claim 1 wherein the composition has a pH in the range of 4 to 9.

7. A composition as in claim 1 wherein the composition has an osmolality of 200 to 450 milliosmoles per kilogram (mOsm/kg).

8. An ophthalmic composition comprising a compound of formula A: 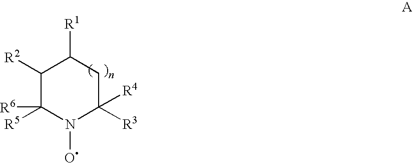
wherein the compound of formula A is selected from the group consisting of: 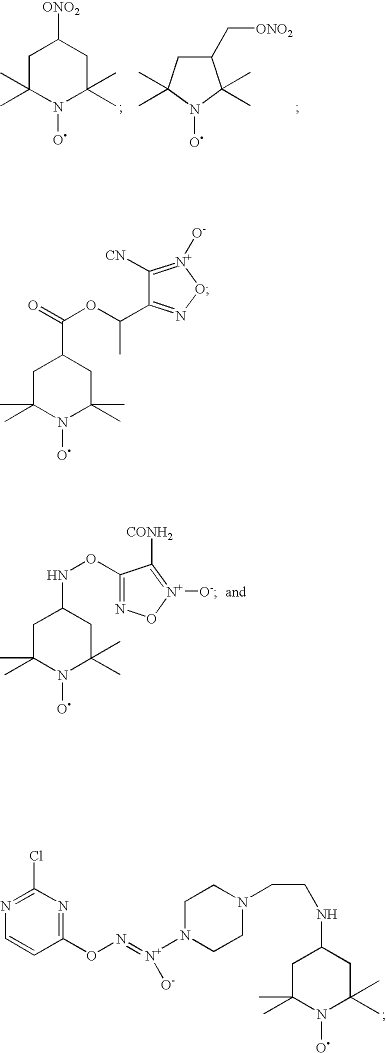
and
a suitable ophthalmic vehicle.

9. A composition as in claim 8 wherein the composition is formulated for topical application and is disposed with an eye dropper and wherein the composition further includes an antimicrobial agent, a surfactant, a tonicity agent or a combination thereof.

10. A composition as in claim 8 wherein the composition has a pH in the range of 5.5 to 8.5.

11. A composition as in claim 8 wherein the composition has an osmolality of 240 to 360 mOsm/kg.

12. An ophthalmic composition comprising a compound of formula A: 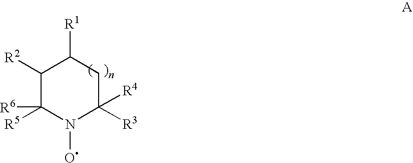
wherein the compound of formula A is selected from the group consisting of: 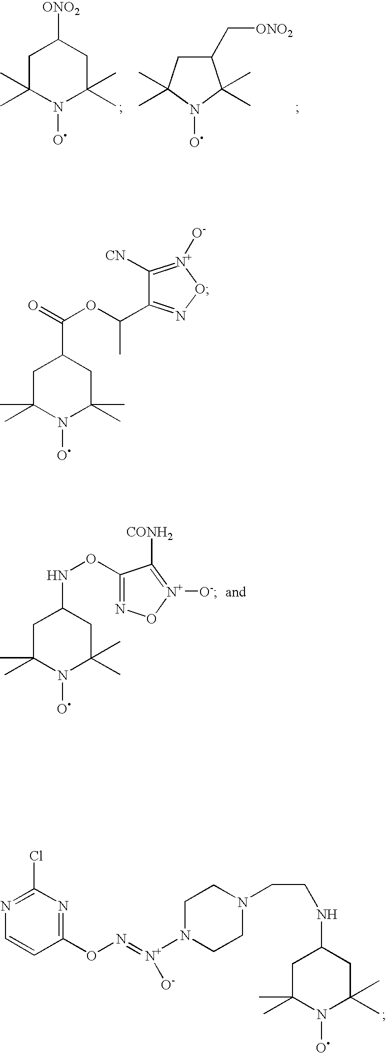
and
a suitable ophthalmic vehicle, wherein:
i. the composition is formulated for topical application and is disposed with an eye dropper;
ii. the composition further includes an antimicrobial agent, a surfactant, and a tonicity agent;
iii. the composition has a pH in the range of 6.0 to 7.8; and
iv. the composition has an osmolality of 240 to 360 mOsm/kg.

13. A method of reducing intraocular pressure or treating age-related macular degeneration, diabetic retinopathy, or uveitis, comprising:
topically administering to a human in need of such treatment the composition of claim 1.

14. A method of reducing intraocular pressure, comprising:
testing a human or other mammal to determine if the human or other mammal has elevated intraocular pressure;
topically administering to the human or other mammal a therapeutically effective amount of the compositions of claim 1.

15. A method of reducing intraocular pressure or treating age-related macular degeneration, diabetic retinopathy, or uveitis, comprising:
topically administering to a human in need of such treatment the composition of claim 8.

16. A method of reducing intraocular pressure, comprising:
testing a human or other mammal to determine if the human or other mammal has elevated intraocular pressure; and
topically administering to the human or other mammal a therapeutically effective amount of the compositions of claim 8.

17. A method of reducing intraocular pressure or treating age-related macular degeneration, diabetic retinopathy, or uveitis, comprising:
topically administering to a human in need of such treatment the composition of claim 12 as eye drops from the eye dopper.

18. A method of reducing intraocular pressure, comprising:
testing a human or other mammal to determine if the human or other mammal has elevated intraocular pressure; and
topically administering to the human or other mammal a therapeutically effective amount of the compositions of claim 12 as eye drops from the eye dropper.
